(19) United States
(12) Patent Application Publication
(10) Pub. No.: US 2016/0024175 A1
(43) Pub. Date: Jan. 28, 2016

(54) CHEMOTHERAPY-RESISTANT IMMUNE CELLS

(71) Applicant: Baylor College of Medicine, Houston, TX (US)

(72) Inventors: Kevin Kwong-Hon Chow, Houston, TX (US); Zhongzhen Yi, Houston, TX (US); Donald R. Shaffer, Boston, MA (US); Stephen M. G. Gottschalk, Houston, TX (US)

(21) Appl. No.: 14/773,416

(22) PCT Filed: Mar. 10, 2014
(86) PCT No.: PCT/US2014/022786

§ 371 Date: Sep. 8, 2015

Related U.S. Application Data
(60) Provisional application No. 61/775,668, filed on Mar. 10, 2013.

(51) Int. Cl. C07K 14/705; C07K 14/725; A61N 5/10; A61K 38/45; A61K 38/17; A61K 45/06; C12N 9/10; A61K 31/4188

(52) U.S. Cl. CPC C07K 14/70521; C12N 9/1007; C07K 14/7051; A61K 31/4188; A61K 38/45; A61K 38/1774; A61K 45/06; A61N 5/10; A61K 48/00

(57) ABSTRACT

Embodiments of the disclosure include compositions and methods useful for treating cancers that are sensitive to a chemotherapy, such as temozolomide. The methods allow effective cell immunotherapy to be used with chemotherapy when the cell immunotherapy is susceptible to being rendered ineffective by the chemotherapy. In specific aspects, the cancer is being treated by temozolomide (TMZ) and tumor antigen-specific T-cells that are resistant to TMZ.

This application claims priority to U.S. Provisional Patent Application Ser. No. 61/775,668, filed Mar. 10, 2013, which is incorporated by reference herein in its entirety.

### TECHNICAL FIELD

Embodiments of the disclosure concern at least the fields of cell biology, immunology, molecular biology, and medicine, including cancer medicine.

### BACKGROUND

While Temozolomide (TMZ) is FDA approved for anaplastic astrocytoma and glioblastoma multiforme (GBM), it is currently being evaluated for a broad range of malignancies including pediatric cancers such as neuroblastoma, and common adult cancers such as prostate cancer and melanoma. This disclosure is focused on generating immune cells that are resistant to TMZ so that they can be given to patients, who receive TMZ. Embodiments of the disclosure concern GBM, but the disclosure can be applied to any cancer that is treated with TMZ. TMZ was shown in a large multicenter phase III trial to significantly improve the overall survival of glioblastoma multiforme (GBM) patients when given in combination with radiation therapy as compared to radiotherapy alone. As such, TMZ is now the preferred chemotherapeutic agent for patients with newly diagnosed GBM. Administration of TMZ follows surgical resection of the tumor. Specifically, the recommended schedule is to give TMZ (75 mg/m² daily up to 49 days) concomitant with involved-field radiotherapy (60 Gy in daily 30 fractions) followed by TMZ alone (150 to 200 mg/m² daily for five days, every 28 days). While in the original study, TMZ monotherapy was given for up to six months, most patients currently receive up to 12 months as long as they remain in remission or do not develop progressive disease.

TMZ works by methylating the O⁶ position of guanine residues on DNA. These lesions, however, are not immediately cytotoxic and can be repaired by a DNA repair protein called O⁶-methylguanine-DNA methyltransferase (MGMT). One MGMT molecule can catalyze the transfer of one O⁶-methyl group to a cysteine residue in the MGMT protein, thereby repairing the DNA lesion and inactivating the MGMT. The capacity to rapidly repair multiple lesions therefore depends on the amount of MGMT expressed by the cell. Once depleted, MGMT must be newly synthesized for removal of additional O⁶-methylguanine adducts. If left unrepaired, O⁶-methylguanine becomes mispaired with thymine leading to gapped DNA and double strand breaks during DNA replication that ultimately triggers cell death via the apoptotic pathway.

Indeed, patients who had GBM tumors with highly methylated, and therefore silenced, MGMT promoters had better responses to TMZ therapy presumably because of compromised DNA repair. Among patients receiving TMZ, the group with unmethylated MGMT promoters had a median overall survival of 12.7 months while the group with methylated MGMT promoters had a survival of 21.7 months. Clearly, these results point to the importance of MGMT in determining susceptibility to TMZ and indicate the potential for identifying patients that are most likely to respond to TMZ therapy. For patients with unmethylated MGMT promoters, the addition of O⁶-benzylguanine (O⁶-BG), an inhibitor of MGMT, to an alkylating agent can improve antitumor activity; however, this strategy is associated with dose-limiting hematopoietic toxicity.

To improve outcome, one strategy may be to combine cell-based immunotherapy with chemotherapy for GBM. Like most chemotherapeutic agents, the cytotoxic effects of TMZ are relatively nonspecific, and have their greatest effect on rapidly dividing cells. This toxicity against rapidly dividing cells accounts for its antitumor activity as well as its adverse effects including alopecia, gastrointestinal upset, and myelosuppression. While TMZ in general is well tolerated at the used dosing schedule, myelosuppression including profound lymphopenia is one of its major side effects. Studies have shown that giving chemotherapy to lymphodeplete patients prior to T-cell immunotherapy improved antitumor activity by providing a better environment for the expansion of the adoptively transferred cells. Chemotherapy may also directly enhance the immunogenicity of tumor cells and make them more susceptible to killing by adoptively transferred T cells.

In these studies, the timing of administering the chemotherapy and immunotherapy were critical, since it is the initial treatment with chemotherapy that primes an effective immunotherapeutic response. In the case of newly-diagnosed GBM, however, where the standard of care is to give TMZ repeatedly for up to 12 months after surgery, choosing when to initiate adoptive T-cell transfer may be problematic: while giving T cells at the time of minimal residual disease would be ideal, infusing T cells while a patient is receiving TMZ may prevent robust proliferation of the adoptively transferred T cells in vivo, thus undermining an effective immunotherapeutic response.

Therefore, there is a need in the art to improve therapies associated with TMZ in patients having GBM, and the present disclosure satisfies such a need. Embodiments of the disclosure are also applicable to those that are being treated with TMZ for other cancers or for chemotherapies other than TMZ.

### BRIEF SUMMARY

Embodiments of the disclosure concern combination therapies for individuals in need of cancer treatment. In particular embodiments, the combination therapies encompass one therapy in the combination that is susceptible to being rendered ineffective (or being reduced in activity) by another therapy in the combination. In specific embodiments, the combination therapies include immunotherapy and chemotherapy, in which the chemotherapy is capable of inhibiting not only its target cancer cells in the individual but also the immune cells being employed as a co-therapy for the individual.

Embodiments of the present disclosure address the dilemma of needing to provide immune cells (T-cells, NK-cells, or NKT-cells, for example) while a patient is receiving chemotherapy without preventing robust proliferation of the adoptively transferred immune cells in vivo that would weaken an effective immunotherapeutic response. Immune cells can either be unspecific or recognize, for example, antigens expressed on tumor or the supporting tumor stroma. Embodiments of the disclosure overcome this limitation, by providing immune cells that are resistant to the toxic effects of the chemotherapy such that they can be administered concomitantly with the chemotherapeutic drug.

In particular embodiments, there are cells that comprise both a chemotherapy-resistance moiety, e.g., a TMZ-resistance moiety, and that comprise an antigen recognition moiety. The cells may harbor the chemotherapy-resistance moiety and the antigen recognition moiety as separate polypeptides. The cells may harbor polynucleotides that encode the chemotherapy-resistance moiety and the antigen recognition moiety, including one polynucleotide that encodes both moieties or separate polynucleotides for each moiety. The chemotherapy-resistance moiety may be specific for one or more chemotherapies. In some cases, the chemotherapy-resistance moiety is O⁶-methylguanine-DNA methyltransferase (MGMT), multidrug resistance gene (MDR), or 5′-nucleotidase II (NT5C2). The chemotherapy may be any type of chemotherapy, including nucleoside-analog chemotherapy drugs, alkylating agents, antimetabolites, antibiotics (including anthracyclines), topoisomerase inhibitors, mitotic inhibitors, differentiating agents, hormone therapy agents, and so forth.

The antigen recognition moiety may be of any kind, so long as it is capable of immunologically recognizing an antigen, including a tumor antigen. The antigen may be on tumor cells, secreted from tumor cells, on cells in the tumor stroma, or in the tumor stroma. The antigen recognition moiety, in specific embodiments, is a chimeric antigen receptor (CAR), an αβTCR, or an engager molecule.

Thus, in particular embodiments, the present disclosure is directed to methods and compositions related to immunotherapy that comprises cell therapy in addition to chemotherapy. In particular embodiments, the cell therapy is for an individual in need of cell therapy, such as a mammal, including a human. The cell therapy may be suitable for any medical condition, although in specific embodiments the cell therapy is for cancer, including cancer that is treatable by TMZ, for example. The cancer that is treatable by TMZ may be any kind, but in specific embodiments the cancer is GBM, other brain tumors including anaplastic astrocytoma, neuroblastoma, prostate cancer, or melanoma, for example. In addition TMZ can be used to selectively expand TMZ-resistant, tumor-specific T cells in cancer patients, even when the tumor is not sensitive to TMZ. Thus this approach could be applied to a broad range of malignancies including breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervical cancer and kidney cancer, lung cancer, gastric cancer, cancer of the small intestine, liver cancer, pancreas cancer, gall bladder cancer, cancers of the bile duct, esophagus cancer, cancer of the salivatory glands and cancer of the thyroid gland, and hematological malignancies including, but not limited to, leukemia, lymphoma, multiple myeloma, and myelodysplastic syndromes.

Specific embodiments of the disclosure concern the combination of immunotherapy with chemotherapy for GBM utilizing TMZ-resistant immune cells, such as T-cells that are genetically modified to express chimeric antigen receptors (CARs). Although others have expressed MGMT in hematopoietic progenitors to make them TMZ-resistant, allowing them to be used for in vivo selection by TMZ treatment following hematopoietic stem cell transplantation, the present disclosure instead employs genetically modified immune cells to overexpress MGMT in order to facilitate the combination of those immune cells with standard chemotherapy for GBM.

In embodiments of the disclosure, there is a polynucleotide comprising a sequence that encodes O⁶-methylguanine-DNA methyltransferase (MGMT). In embodiments of the disclosure, there is a polynucleotide comprising a sequence that encodes MGMT and a sequence that encodes a chimeric antigen receptor (CAR); the respective sequences may or may not be on the same polynucleotide molecule. In specific embodiments, the MGMT and the CAR are expressed as gene products that are separate polypeptides. In certain cases, the CAR is specific for a tumor antigen, such as a tumor antigen that is present on a cancer cell, wherein the cancer is treatable by Temozolomide (TMZ). In certain cases, the tumor antigen is expressed in glioblastoma multiforme (GBM) cells, melanoma cells, lymphoma cells, breast cancer cells, prostate cancer cells, neuroblastoma cells, or cells of any other cancer that is being treated with a TMZ-comprising regimen. In particular embodiments, the tumor antigen is any antigen expressed in the tumor and/or associated tumor stroma including EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracelluar matrix of tumors, such as oncofetal variants of fibronectin, tenascin, or necrotic regions of tumors, viral-associated antigens expressed by the tumor, or other tumor-associated antigens that are identified through genomic analysis and/or differential expression studies of tumors.

In embodiments of the disclosure there is an expression vector, comprising at least one of any polynucleotides of the disclosure. In specific embodiments, the vector is a viral vector, such as a retroviral vector, lentiviral vector, adenoviral vector, or adeno-associated viral vector.

In embodiments of the disclosure, there is a cell comprising at least one expression vector of the disclosure. The cell may be further defined as an immune system cell. The cell may be further defined as a T-cell, NK-cell, or NKT-cell, or any other immune cell with an effector function. The cell may be further defined a T cell.

In particular embodiments of the disclosure, there is combination therapy for an individual that has been diagnosed with cancer, wherein the combination therapy may comprise two, three, or more cancer therapeutics. The therapies in the combination therapy may be administered to the individual at the same or at different times. The individual's cancer may have one, two, or more specific tumor antigens on the surface of the cancer cells to which the immunotherapy for the individual is targeted. In particular aspects the tumor antigen is HER2, although in some cases it is another antigen expressed by cancer cells or their supporting stroma. In specific aspects, the tumor antigen HER2 is present on GBM cells, other brain tumor, breast cancer, lung cancer, sarcoma, ovarian, for example. Thus, a particular cell may have two or more antigen recognizing moieties, each specific for a different antigen.

In embodiments of the disclosure, there is a method of treating cancer that is treatable with a chemotherapy, comprising the step of delivering a therapeutically effective amount of any of the cells of the disclosure to an individual that is receiving, has received, or will receive the chemotherapy. In embodiments of the disclosure, there is a method of treating cancer that is treatable with a particular chemotherapy (such as TMZ), comprising the step of delivering a therapeutically effective amount of any of the cells of the disclosure to an individual that is receiving, has received, or will receive the chemotherapy. In specific embodiments, any method of the disclosure may be further defined including delivery of a therapeutically effective amount of the chemotherapy to the individual. In specific cases, the cells and the chemotherapy are delivered concomitantly, are delivered at separate times, are delivered by the same delivery route, or are delivered by different delivery routes. In particular aspects of the disclosure for individuals being treated with TMZ, when an individual has unmethylated MGMT promoters, the individual may be provided with an effective amount of O⁶-benzylguanine (O⁶-BG) in addition to other method steps of the disclosure. In some cases, any method of the disclosure further comprises the step of diagnosing the cancer of the individual, for example in an individual suspected of having a cancer that is treatable with a chemotherapy, such as TMZ.

In particular aspects of any method of the disclosure, the cancer is glioblastoma multiforme, melanoma, lymphoma, breast cancer, prostate cancer, neuroblastoma, or any other cancer for which is being treated with a TMZ-based regimen. The cancer may be metastatic cancer. In specific embodiments, the CAR is specific for any antigen expressed in the tumor and/or associated tumor stroma. Any method of the disclosure may further comprise the step of delivering an additional cancer treatment to the individual, such as treatment that comprises surgery, chemotherapy, immunotherapy, radiation, hormone therapy, or a combination thereof.

In embodiments of the disclosure, there is a kit comprising one or more of any polynucleotide of the disclosure, one or more of any expression vector of the disclosure, and/or one or more of any cells of the disclosure, in addition to or as an alternative to also including reagents and compositions suitable for generating said polynucleotides, vectors, and/or cells. The chemotherapy may be included in the kit, in certain embodiments.

The cancer being treated by methods and compositions of the disclosure may be of any kind and of any stage. The individual may be of any age or either gender. In specific embodiments, the individual is known to have cancer, is at risk for having cancer, or is suspected of having cancer. The cancer may be a primary or metastatic cancer, and the cancer may be refractory to treatment.

In one embodiment, there are one or more polynucleotides that comprise sequence that encodes a chemotherapy-resistance gene product and that comprises sequence that encodes an antigen recognition moiety. In a specific embodiment, the chemotherapy resistance gene product is resistant to a nucleoside-analog chemotherapy drug, alkylating agent, antimetabolite, antibiotic, topoisomerase inhibitor, mitotic inhibitor, differentiating agent, or hormone therapy agent. In specific embodiments, the chemotherapy-resistance gene product is O⁶-methylguanine-DNA methyltransferase (MGMT), multidrug resistance gene (MDR), or 5′-nucleotidase II (NT5C2). In particular embodiments, the antigen recognition moiety is a tumor antigen recognition moiety, such as a chimeric antigen receptor (CAR), engineered αβTCR, or engager molecule. In a certain embodiment, the tumor antigen is an antigen expressed in or from cells of the tumor and/or in the associated tumor stroma. In some cases, the CAR is specific for a tumor antigen that is present on a cancer cell and wherein the cancer is treatable by Temozolomide (TMZ). In particular embodiments, the cancer cells are glioblastoma multiforme (GBM) cells, anaplastic astrocytoma cells, melanoma cells, lymphoma cells, breast cancer cells, prostate cancer cells, or neuroblastoma cells. In specific embodiments, the tumor antigen is selected from the group consisting of EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, and VEGF receptors. In certain embodiments, the chemotherapy-resistance gene product and the antigen recognition moiety are expressed as gene products that are separate polypeptides. In certain embodiments, the polynucleotide that comprises sequence that encodes a chemotherapy-resistance gene product and the polynucleotide that comprises sequence that encodes an antigen recognition moiety are separate polynucleotides.

In one embodiment, there is an expression vector, comprising a polynucleotide of the disclosure. In specific embodiments, the vector is a viral vector, such as a retroviral vector, lentiviral vector, adenoviral vector, or adeno-associated viral vector. In some embodiments, there is a cell comprising an expression vector of the disclosure. In specific embodiments, the cell is an immune system cell, such as a T-cell, NK-cell, or NKT-cell.

In one embodiment, there is a method of treating cancer that is treatable with a chemotherapy, comprising the step of delivering a therapeutically effective amount of the cells of the disclosure to an individual that is receiving, has received, or will receive the chemotherapy. In a specific embodiment, the method further comprises the step of delivering a therapeutically effective amount of the chemotherapy to the individual. In specific embodiments, the cells and the chemotherapy are delivered to the individual concomitantly. In certain aspects, the cells and the chemotherapy are delivered to the individual at separate times. In particular embodiments, the cells are delivered prior to the chemotherapy. In some cases, the chemotherapy is delivered prior to the cells. In specific embodiments, the cells and the chemotherapy are delivered by the same delivery route, although the cells and the chemotherapy may be delivered by different delivery routes. In specific embodiments, the cancer is glioblastoma multiforme, melanoma, lymphoma, breast cancer, prostate cancer, or neuroblastoma. In some cases, the cancer is metastatic cancer. The chemotherapy may be TMZ, in at least some cases. In specific embodiments, when the antigen recognition moiety is a CAR, the CAR is specific for HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α2, IL-11 receptor R α, MUC1, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, B7-H3, NCAM, VEGF receptors, 5T4, Fetal AchR, NKG2D ligands, Glypican-3, CD44v6, TEM1, or TEM8. In some embodiments, methods of the disclosure further comprise the step of delivering an additional cancer treatment to the individual. In some embodiments, the additional cancer treatment comprises surgery, chemotherapy, immunotherapy, radiation, hormone therapy, or a combination thereof. In specific embodiments, when the individual has unmethylated MGMT promoters, the individual is provided with an effective amount of O⁶-benzylguanine (O⁶-BG). In certain embodiments, the method further comprises the step of diagnosing the cancer of the individual.

In one embodiment, there is a kit comprising a polynucleotide of the disclosure, an expression vector of the disclosure, and/or the cells of the disclosure.

In one embodiment, there is a method of treating cancer in an individual, comprising the step of administering to the individual a therapeutically effective amount of the cells of the disclosure, wherein the cancer is sensitive to the therapy for which the cells are resistant. In a specific embodiment, the cancer is selected from the group consisting of breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervical cancer and kidney cancer, lung cancer, gastric cancer, cancer of the small intestine, liver cancer, pancreas cancer, gall bladder cancer, cancers of the bile duct, esophagus cancer, cancer of the salivatory glands and cancer of the thyroid gland, and hematological malignancies including, but not limited to, leukemia, lymphoma, multiple myeloma, and myelodysplastic syndromes.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows that combining TMZ with HER2.CAR T cells enhances tumor killing. U373.eGFP.FFLuc cells were cocultured with either non-transduced (NT) or HER2.CAR T cells with TMZ (62.5 and 125 μM) or without TMZ (DMSO). Tumor killing was evaluated by fluorescence microscopy. Only the groups receiving both TMZ and HER2.CAR T cells showed complete tumor killing.

FIG. 2 demonstrates that TMZ inhibits T-cell expansion. 1×10⁶ HER2.CAR T cells were plated on day 1 with 100 U/ml of IL-2 and treated with 100 μM TMZ. Every three days, the T cells were counted, given fresh IL-2, and retreated with TMZ. While HER2.CAR T cells expanded in the absence of TMZ, treatment with 100 μM TMZ effectively prevented T-cell expansion in vitro (mean±s.d.; n=4 donors; *p<0.05).

FIGS. 3A-3C show the generation of TMZ-resistant HER2.CAR T cells. MGMT was cloned into an SFG retroviral vector with a second generation HER2.CAR (3A). This HER2.MGMT construct was used to generate T cells that expressed both the HER2.CAR and functional MGMT protein (HER2.MGMT T cells). A second construct containing a cysteine (C) to alanine (A) amino acid substitution in the MGMT sequence was used to generate control T cells that express the HER2.CAR, and nonfunctional MGMT protein (HER2.MGMTCA T cells). HER2.CAR expression was detected by staining T cells with an FAB antibody (3B), and MGMT expression was determined by quantitative RT-PCR (3C). Both HER2.MGMT and HER2.MGMTCA T cells express comparable levels of HER2.CAR (data representative of 4 donors) and MGMT mRNA (mean±s.d.; n=4 donors; data normalized to GAPDH).

FIG. 4 provides that T cells transduced with active MGMT expand in the presence of TMZ. 1×10⁶ HER2.MGMT or HER2.MGMTCA T cells were plated on day 1 with 100 U/ml of IL-2 and treated with 100 μM TMZ. Every three days, the T cells were counted, given fresh IL-2, and retreated with TMZ. While both HER2.MGMT and HER2.MGMTCA T cells could expand in the absence of TMZ, only HER2.MGMT T cells could expand in the presence of TMZ (mean±s.d.; n=4 donors; *p<0.05 for HER2.MGMT+TMZ vs. HER2.MGMTCA+TMZ from day 7 to day 22).

FIG. 5 shows that HER2.MGMT T cells undergo less apoptosis in the presence of TMZ. 1×10⁶ non-transduced (NT), HER2.MGMTCA, or HER2.MGMT T cells were plated with 100 U/ml of IL-2 and treated with 100 or 200 μM TMZ on day 0. The cells were retreated with IL-2 and TMZ on day 3, and analyzed for annexin and 7-AAD by FACS on day 7. All groups showed little apoptosis in the absence of TMZ. HER2.MGMT T cells underwent less apoptosis in the presence of TMZ than NT or HER2.MGMTCA T cells.

FIG. 6 demonstrates that pretreatment of HER2.MGMT T cells with TMZ does not impair tumor killing. 5×10⁵ non-transduced, HER2.MGMTCA, or HER2.MGMT T cells were treated with IL-2±TMZ on day 0 and day 3. On day 7, 5×10⁵ U373.eGFP.FFLuc were added to the T cells. On day 9, the T cells were removed and the tumor cells were imaged by fluorescence microscopy. HER2.MGMT T cells were able to completely kill the tumor cells even after being pretreated with TMZ, whereas the HER2.MGMTCA T cells could not.

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

### I. Chemotherapy Resistance Moiety

In embodiments of the disclosure, the immune cells for the immunotherapy comprise one or more chemotherapy resistance moieties. The chemotherapy resistance moiety, in specific aspects, includes at least one of a gene product encoded by a chemotherapy resistance gene. In some cases, a particular cell comprises two or more chemotherapy resistance moieties, and in instances wherein there are multiple chemotherapy resistance moieties in the same cell, they may be of the same type of moiety (for example, provide resistance to the same type of chemotherapy), or they may be different types of moieties (for example, provide resistance to different types of chemotherapy). In alternative embodiments, a plurality of immune cells are delivered to an individual wherein in the plurality there is a mixture of cells including a cell that comprises a first chemotherapy resistance moiety and an antigen recognition moiety and another cell in the plurality that comprises a second chemotherapy resistance moiety and an antigen recognition moiety.

In specific embodiments, the chemotherapy resistance moiety is O⁶-methylguanine-DNA methyltransferase (MGMT), multidrug resistance gene (MDR), 5′-nucleotidase II (NT5C2). The skilled artisan will recognize that the chemotherapy resistance moiety will be tailored to the chemotherapy being provided to the individual. For example, when TMZ is being delivered, or is to be delivered, to the individual, the immune cell will be modified to incorporate MGMT.

### II. Antigen Recognition Moiety

In embodiments of the disclosure, the immune cells for the immunotherapy comprise one or more antigen recognition moieties.

The antigen recognition moiety, in specific aspects, includes at least one of a CAR, αβTCR, or an engager molecule. In some cases, a particular cell comprises two or more antigen recognition moieties, and in instances wherein there are multiple antigen recognition moieties in the same cell, they may be of the same type of moiety (for example, both are CARs), or they may be different types of moieties (for example, one CAR and one engager).

In alternative embodiments, a plurality of immune cells are delivered to an individual wherein in the plurality there is a mixture of cells including a cell that comprises a first antigen recognition moiety and a chemotherapy resistance moiety and another cell in the plurality that comprises a second antigen recognition moiety and a chemotherapy resistance moiety.

A. Chimeric Antigen Receptors (CARs)

In some cases, the immune cells are modified to express a CAR. Genetic engineering of human T lymphocytes to express tumor-directed chimeric antigen receptors (CAR) can produce antitumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the disclosure there are CTLs that are modified to comprise at least a CAR.

In particular cases, the cytotoxic T lymphocytes (CTLs) include a receptor that is chimeric, non-natural and engineered at least in part by the hand of man. In particular cases, the engineered chimeric antigen receptor (CAR) has one, two, three, four, or more components, and in some embodiments the one or more components facilitate targeting or binding of the T lymphocyte to the tumor antigen-comprising cancer cell. In specific embodiments, the CAR comprises an antibody for the tumor antigen, part or all of a cytoplasmic signaling domain, and/or part or all of one or more co-stimulatory molecules, for example endodomains of co-stimulatory molecules. In specific embodiments, the antibody is a single-chain variable fragment (scFv). In certain aspects the antibody is directed at target antigens on the cell surface of cancer cells that are treatable by TMZ, for example. In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD27, CD28, 4-1BB, and OX40 or the signaling components of cytokine receptors such as IL7 and IL15. In particular embodiments, co-stimulatory molecules are employed to enhance the activation, proliferation, and cytotoxicity of T cells produced by the CAR after antigen engagement. In specific embodiments, the co-stimulatory molecules are CD28, OX40, and 4-1BB and cytokine and the cytokine receptors are IL7 and IL15.

The CAR may be first generation, second generation, or third generation (CAR in which signaling is provided by CD3ζ together with co-stimulation provided by CD28 and a tumor necrosis factor receptor (TNFr), such as 4-1BB or OX40), for example. The CAR may be specific for HER2 and it may include other CARs, such as those specific for CD19, CD20, CD22, Kappa or light chain, CD30, CD33, CD123, CD38, ROR1, ErbB2, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, viral-associated antigens expressed by the tumor, or other tumor-associated antigens that are identified through genomic analysis and or differential expression studies of tumors.

In particular cases the CAR is specific for HER2, and in certain embodiments, the present disclosure provides chimeric T cells specific for HER2 by joining an extracellular antigen-binding domain derived from the HER2-specific antibody to cytoplasmic signaling domains derived from the T-cell receptor ζ-chain, with the endodomains of the exemplary costimulatory molecules CD28, for examples. This CAR is expressed in human T cells and the targeting of HER2-positive cancers is encompassed in the disclosure. In some cases, the same cell comprises a CAR specific for HER2 and a CAR specific for another antigen that may or may not be present cancer cells treatable by TMZ. In some cases, the same cell comprises a CAR specific for HER2 and a CAR specific for another antigen that may or may not be present on glioblastoma cells.

In particular embodiments the CAR is encoded on an expression vector that also encodes the chemotherapy resistance moiety. The vector is bicistronic, in particular embodiments. The CAR coding sequence may be configured 5′ or 3′ to the chemotherapy resistance moiety coding sequence. The expression of the CAR and the chemotherapy resistance moiety may be under the direction of the same or different regulatory sequences.

B. αβT-Cell Receptor (TCR)

In certain cases, the immune cells of the disclosure include an αβTCR, including an engineered αβTCR. In particular embodiments, the αβTCR is specific for a peptide presented on a HLA molecule expressed by a tumor cell. Peptides are derived from tumor antigens and examples, but not limited to, include peptides derived from gp100, mage family members, NY-ESO-1, PRAME, and WT1.

C. Engager Molecules

In particular embodiments, the immune cells of the disclosure comprise engager molecules as their antigen recognition moiety.

In particular aspects, the engager molecule as the antigen recognition moiety comprises an activation domain that binds to an activation molecule on an immune cell surface (or an engineered immune cell surface), and an antigen recognition domain that binds to a target cell antigen, e.g., an antigen expressed on the surface of a tumor cell or cancer cell.

The engager may be bipartite (e.g., comprising an activation domain and antigen recognition domain that may optionally be joined by a linker), or may be tripartite or multipartite (e.g., comprise one or more activation domains and/or antigen recognition domains, or other domains). In specific embodiments, the activation domain of the engager is or comprises an antibody or an antigen-binding fragment or portion thereof, e.g., a single chain variable fragment (scFv). On other specific embodiments, the antigen recognition domain is or comprises an antibody or an antigen-binding fragment or portion thereof, e.g., a monoclonal antibody or an scFv, or it may comprise ligands, peptides, soluble T-cell receptors, or combinations thereof. In certain embodiments, the activation domain and antigen recognition domain are joined by a linker, e.g., a peptide linker.

The skilled artisan recognizes that immune cells have different activating receptors, and the engager will be tailored to the cell being activated. For example CD3 is an activating receptor on T-cells, whereas CD16, NKG2D, or NKp30 are activating receptors on NK cells, and CD3 or an invariant TCR are the activating receptors on NKT-cells. Engager molecules that activate T-cells may therefore have a different activation domain than engager molecules that activate NK cells. In specific embodiments, e.g., wherein the immune cell is a T-cell, the activation molecule is one or more of CD3, e.g., CD3γ, CD3δ or CD3ε; or CD27, CD28, CD40, CD134, CD137, and CD278. In other specific embodiments, e.g., wherein the immune cell is a NK cell, the activation molecule is CD16, NKG2D, or NKp30, or wherein the immune cell is a NKT-cell, the activation molecule is CD3 or an invariant TCR.

In certain other embodiments, the engager additionally comprises one or more accessory domains, e.g., one or more of a cytokine, a costimulatory domain, a domain that inhibits negative regulatory molecules of T-cell activation, or a combination thereof. In specific embodiments, the cytokine is IL-15, IL-2, and/or IL-7. In other specific embodiments, the costimulatory domain is CD27, CD80, CD86, CD134, or CD137. In other specific embodiments, the domain that inhibits negative regulatory molecules of T-cell activation is PD-1, PD-L1, CTLA4, or B7-H4.

### III. Host Cells Comprising a Chemotherapy Resistance Moiety and an Antigen Recognition Moiety

Host cells of the disclosure are immune cells that are modified to express at least a chemotherapy resistance moiety and an antigen recognition moiety. As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the disclosure, a host cell is a T cell, including a cytotoxic T cell (also known as TC, Cytotoxic T Lymphocyte, CTL, T-Killer-cell, cytolytic T-cell, CD8+ T-cells or killer T cell); NK cells, NKT cells, and other immune cells that can elicit an effector function are also encompassed in the disclosure.

In certain embodiments, it is contemplated that RNAs or proteinaceous sequences may be co expressed with other selected RNAs or proteinaceous sequences in the same cell, such as the same CTL. Co-expression may be achieved by co-transfecting the CTL with two or more distinct recombinant vectors. Alternatively, a single recombinant vector may be constructed to include multiple distinct coding regions for RNAs, which could then be expressed in CTLs transfected with the single vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells, in relation to the individual that is receiving the cells.

In many situations one may wish to be able to kill the modified CTLs, where one wishes to terminate the treatment, the cells become neoplastic, in research where the absence of the cells after their presence is of interest, or other event. For this purpose one can provide for the expression of certain gene products in which one can kill the modified cells under controlled conditions, such as inducible suicide genes.

Expression vectors that encode the antigen recognition moiety (such as HER2 CARs) and chemotherapy resistance moiety (such as MGMT) can be introduced into the cells as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the antigen recognition moiety and the chemotherapy resistance moiety and optionally another gene, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

### IV. Pharmaceutical Compositions

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration or for direct injection into a cancer. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A preferred dosage for administration might be in the range of 0.24 μg to 48 mg, preferably 0.24 μg to 24 mg, more preferably 0.24 μg to 2.4 mg, even more preferably 0.24 μg to 1.2 mg and most preferably 0.24 μg to 240 mg units per kilogram of body weight per day. Particularly preferred dosages are recited herein below. Progress can be monitored by periodic assessment. Multiple administrations of the cells are encompassed in the methods of the disclosure. An example of a cell dose might be in the range of 1×10⁷ to 1×10¹⁰ cells given at 6 to 8 week dosing intervals. Other intervals that may be used are 2 to 10, 2 to 8, 2 to 6, 4 to 10, 4 to 8, 4 to 6 weeks, and so forth. In a specific embodiment, the cells are provided once to the individual.

The compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes an engager molecule as described herein and/or regulatory elements therefor.

In particular embodiments, there are one or more apparatuses in the kit suitable for extracting one or more samples from an individual. The apparatus may be a syringe, scalpel, and so forth.

In particular aspects, the kit comprises the cell therapy of the disclosure and also the chemotherapy for which the cells are immune. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### V. Therapeutic Uses of Host T-Cells Expressing Chemotherapy Resistance Moieties and Antigen Recognition Moieties

By way of illustration, cancer patients or patients susceptible to cancer or suspected of having cancer may be treated as described herein. Immune cells modified as described herein may be administered to the individual and retained for extended periods of time. The individual may receive one or more administrations of the cells. In some embodiments, the genetically modified cells are encapsulated to inhibit immune recognition and placed at the site of the tumor.

In various embodiments the expression constructs, nucleic acid sequences, vectors, host cells and/or pharmaceutical compositions comprising the same are used for the prevention, treatment or amelioration of a cancerous disease, such as a tumorous disease. In particular embodiments, the pharmaceutical composition of the present disclosure may be particularly useful in preventing, ameliorating and/or treating cancer, including cancer having solid tumors, for example.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, cells harboring expression constructs, nucleic acid molecules and/or vectors that can administered either alone or in any combination with another therapy, and in at least some aspects, together with a pharmaceutically acceptable carrier or excipient. In certain embodiments, prior to administration of the cells, said nucleic acid molecules or vectors may be stably integrated into the genome of the cells. In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject in need thereof an effective amount of cells harboring an antigen recognition moiety molecule and a chemotherapy resistance molecule, nucleic acid sequence that encodes them, vector(s) that encodes them, as contemplated herein and/or produced by a process as contemplated herein.

Possible indications for administration of the composition(s) of the exemplary modified immune cells are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genito-urinary tract, e.g. ovarian cancer, endometrial cancer, cervix cancer and kidney cancer, lung cancer, gastric cancer, cancer of the small intestine, liver cancer, pancreas cancer, gall bladder cancer, cancers of the bile duct, esophagus cancer, cancer of the salivary glands and cancer of the thyroid gland. In particular aspects, the cancer is treatable by TMZ, for example. Exemplary indications for administration of the composition(s) of cells are cancerous diseases, including any malignancies that express HER2, for example. In addition, it includes malignancies that aberrantly express HER2. The administration of the composition(s) of the disclosure is useful for all stages and types of cancer, including for minimal residual disease, early cancer, advanced cancer, and/or metastatic cancer and/or refractory cancer, for example.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Embodiments relate to a kit comprising one or more immune cells as described herein, a nucleic acid sequence as described herein, a vector as described herein and/or a host as described herein. It is also contemplated that the kit of this disclosure comprises a pharmaceutical composition as described herein above, either alone or in combination with further medicaments to be administered to an individual in need of medical treatment or intervention.

The CTLs that have been modified with the construct(s) are then grown in culture under selective conditions and cells that are selected as having the construct may then be expanded and further analyzed, using, for example; the polymerase chain reaction for determining the presence of the construct(s) in the host cells. Once the modified host cells have been identified, they may then be used as planned, e.g., expanded in culture or introduced into a host organism.

Depending upon the nature of the cells, the cells may be introduced into a host organism, e.g., a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

In particular cases the individual is provided with TMZ and with therapeutic CTLs modified to comprise an expression vector that encodes 1) a CAR specific for HER2 and 2) MGMT. The cells may be delivered at the same time or at different times from the TMZ. The cells and the TMZ may be delivered in the same or separate formulations. The cells and the TMZ may be provided to the individual in separate delivery routes. The cells and/or the TMZ may be delivered by injection at a tumor site or intravenously or orally, for example. Routine delivery routes for such compositions are known in the art.

### VI. Temozolomide

In specific embodiments, Temozolomide (brand names Temodar® and Temodal and Temcad; TMZ) is employed as the chemotherapy to which the immune cells are resistant. TMZ is an oral alkylating agent used for the treatment of at least Grade IV astrocytoma—an aggressive brain tumor, also known as glioblastoma multiforme—as well as for treating melanoma, a form of skin cancer, and other malignancies. It may also be referred to as 3-methyl-4-oxo-3H,4H-imidazo[4,3-d][1,2,3,5]tetrazine-8-carboxamide. Temozolomide may also be used for relapsed Grade III anaplastic astrocytoma and used to treat oligodendroglioma brain tumors. The agent is a derivative of imidazotetrazine, temozolomide is the prodrug of MTIC (3-methyl-(triazen-1-yl)imidazole-4-carboxamide).

Temozolomide is a prodrug that has minimal or no pharmacological activity until it is hydrolyzed in vivo to the active form of 5-(3-methyltriazen-1-yl)imidazole-4-carboxamide (MTIC). Upon administration in vivo, temozolomide undergoes rapid, nonenzymatic hydrolysis at physiological pH through the effect of water at the highly electropositive C4 position of temozolomide causing the ring of temozolomide to open, release carbon dioxide, and generate MTIC.

In some embodiments, the individual has glioblastoma, melanoma, including metastatic melanoma, prostate cancer, neuroblastoma, or relapsed, refractory, or progressive malignant gliomas, for examaple. The individuals receiving the combination therapy of TMZ and T cells of the disclosure may be a child, adolescent, or adult. Doses of TMZ are determine routinely by one of skill in the art and are dependent on the strength of the medicine, the type and stage of cancer being treated, for example.

### VII. Chimeric Antigen Receptors (CARs)-General Concepts

In certain embodiments, the antigen recognition moiety comprises a CAR that is specific for a tumor antigen that is present on a cancer cell that is treatable by a chemotherapy. In specific embodiments, the CAR comprises a bispecific single chain antibody construct that comprises a scFv.

The term “bispecific single chain antibody construct” relates to a construct comprising two antibody derived binding domains. One of the binding domains may comprise variable regions (or parts thereof) of an antibody, antibody fragment or derivative thereof, capable of specifically binding to/interacting with a target antigen. The second binding domain may comprise variable regions (or parts thereof) of an antibody, antibody fragment or derivative thereof, capable of specifically binding to/interacting with an activation molecule, e.g., human CD3 antigen. In specific embodiments, a part of a variable region comprises at least one CDR (“Complementary determining region”), such as at least a CDR1, CDR2, or CDR3 region. The two domains/regions in the single chain antibody construct are preferably covalently connected to one another as a single chain.

An scFv in general contains a VH and VL domain connected by a linker peptide. The secretable engager is composed of a signal peptide (to allow for secretion) from cells, followed by two or more scFvs connected by linker peptides (Lx, Ly, Lz). Linkers may be of a length and sequence sufficient to ensure that each of the first and second domains can, independently from one another, retain their differential binding specificities. Bispecific scFvs can be arranged in different formats: V_{H}α-Lx-V_{L}α-Ly-V_{H}β-Lz-V_{L}β, V_{L}α-Lx-V_{H}α-Ly-V_{H}β-Lz-V_{L}β, V_{L}α-Lx-V_{H}α-Ly-V_{L}β-Lz-V_{H}β, V_{H}α-Lx-V_{L}α-Ly-V_{L}β-Lz-V_{H}β, V_{H}α-Lx-V_{L}β-Ly-V_{H}β-Lz-V_{L}α, V_{H}β-Lx-V_{L}α-Ly-V_{H}α-Lz-V_{L}β, V_{L}α-Lx-V_{H}β-Ly-V_{L}β-Lz-V_{H}α, V_{L}β-Lx-V_{H}α-Ly-V_{L}α-Lz-V_{H}β.

In specific embodiments, the “bispecific single chain antibody construct” to be employed in the composition of the disclosure comprises a bispecific single chain Fv (scFv). Illustrative examples of bispecific single chain molecules are known in the art and are described in WO 99/54440; Mack, J. Immunol. (1997), 158, 3965-3970; Mack, PNAS, (1995), 92, 7021-7025; Kufer, Cancer Immunol. Immunother., (1997), 45, 193-197; Loftier, Blood, (2000), 95, 6, 2098-2103; and Bruhl, J. Immunol., (2001), 166, 2420-2426.

In specific embodiments, an exemplary molecular format of the disclosure provides a polypeptide construct wherein the antibody-derived region comprises one V_{H} and one V_{L} region. In particular embodiments, the intramolecular orientation of the V_{H}-domain and the V_{L}-domain, which are linked to each other by a linker-domain, in the scFv format is not decisive for the recited bispecific single chain constructs. scFvs with both possible arrangements (V_{H}-domain-linker domain-V_{L}-domain; V_{L}-domain-linker domain-V_{H}-domain) are contemplated in particular embodiments of the bispecific single chain construct.

The term “single-chain” as used in accordance with the present disclosure in some embodiments means that first and second domains of the bispecific single chain construct are covalently linked, preferably in the form of a co-linear amino acid sequence encodable by a single nucleic acid molecule.

The term “binding to/interacting with” as used in the context with the present disclosure defines a binding/interaction of at least two “antigen-interaction-sites” with each other. The term “antigen-interaction-site” defines, in accordance with the present disclosure, a motif of a polypeptide that shows the capacity of specific interaction with a specific antigen or a specific group of antigens. The binding/interaction is also understood to define a “specific recognition”. The term “specifically recognizing” means in accordance with this disclosure that the antibody molecule is capable of specifically interacting with and/or binding to at least two amino acids of each of the target molecules as defined herein. The term relates to the specificity of the antibody molecule, i.e. to its ability to discriminate between the specific regions of the human target molecule as defined herein. The specific interaction of the antigen-interaction-site with its specific antigen may result in an initiation of a signal, e.g. due to the induction of a change of the conformation of the antigen, an oligomerization of the antigen, etc. Further, the binding may be exemplified by the specificity of a “key-lock-principle”. Thus, specific motifs in the amino acid sequence of the antigen-interaction-site and the antigen bind to each other as a result of their primary, secondary or tertiary structure as well as the result of secondary modifications of said structure, in some embodiments. The specific interaction of the antigen-interaction-site with its specific antigen may result as well in a simple binding of the site to the antigen.

The term “specific interaction” as used in accordance with the present disclosure means that the bispecific single chain construct does not or essentially does not cross-react with (poly)peptides of similar structures. Cross-reactivity of a panel of bispecific single chain constructs under investigation may be tested, for example, by assessing binding of the panel of bispecific single chain construct under conventional conditions (see, e.g., Harlow and Lane, Antibodies: A Laboratory Manual, Cold Spring Harbor Laboratory Press, 1988 and Using Antibodies: A Laboratory Manual, Cold Spring Harbor Laboratory Press, 1999) to the (poly)peptide of interest as well as to a number of more or less (structurally and/or functionally) closely related (poly)peptides. Only those antibodies that bind to the (poly)peptide/protein of interest but do not or do not essentially bind to any of the other (poly)peptides are considered specific for the (poly)peptide/protein of interest. Examples for the specific interaction of an antigen-interaction-site with a specific antigen comprise the specificity of a ligand for its receptor. The definition particularly comprises the interaction of ligands which induce a signal upon binding to its specific receptor. Examples for corresponding ligands comprise cytokines that interact/bind with/to its specific cytokine-receptors. Another example for said interaction, which is also particularly comprised by said definition, is the interaction of an antigenic determinant (epitope) with the antigenic binding site of an antibody.

The term “binding to/interacting with” may also relate to a conformational epitope, a structural epitope or a discontinuous epitope consisting of two regions of the human target molecules or parts thereof. In context of this disclosure, a conformational epitope is defined by two or more discrete amino acid sequences separated in the primary sequence which come together on the surface of the molecule when the polypeptide folds to the native protein (Sela, (1969) Science 166, 1365 and Layer, (1990) Cell 61, 553-6).

In particular embodiments, the constructs of the present disclosure are also envisaged to specifically bind to/interact with a conformational/structural epitope(s) composed of and/or comprising the two regions of the human CD3 complex described herein or parts thereof as disclosed herein below.

Accordingly, specificity can be determined experimentally by methods known in the art and methods as disclosed and described herein. Such methods comprise, but are not limited to Western blots, ELISA-, RIA-, ECL-, IRMA-, EIA-tests and peptide scans.

The term “antibody fragment or derivative thereof” relates to single chain antibodies, or fragments thereof, synthetic antibodies, antibody fragments, such as a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), and single-domain antibody (sdAb, nanobody), etc., or a chemically modified derivative of any of these. Antibodies to be employed in accordance with the disclosure or their corresponding immunoglobulin chain(s) can be further modified using conventional techniques known in the art, for example, by using amino acid deletion(s), insertion(s), substitution(s), addition(s), and/or recombination(s) and/or any other modification(s) (e.g. posttranslational and chemical modifications, such as glycosylation and phosphorylation) known in the art either alone or in combination. Methods for introducing such modifications in the DNA sequence underlying the amino acid sequence of an immunoglobulin chain are well known to the person skilled in the art; see, e.g., Sambrook et al. (1989).

The term “antibody fragment or derivative thereof” particularly relates to (poly)peptide constructs comprising at least one CDR.

Fragments or derivatives of the recited antibody molecules define (poly)peptides which are parts of the above antibody molecules and/or which are modified by chemical/biochemical or molecular biological methods. Corresponding methods are known in the art and described inter alia in laboratory manuals (see Sambrook et al.; Molecular Cloning: A Laboratory Manual; Cold Spring Harbor Laboratory Press, 2nd edition 1989 and 3rd edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golemis; Protein-Protein Interactions: A Molecular Cloning Manual; Cold Spring Harbor Laboratory Press, 2002).

Variable domains comprised in the herein described bispecific single chain constructs may be connected by additional linker sequences. The term “peptide linker” defines in accordance with the present disclosure an amino acid sequence by which the amino acid sequences of the first domain and the second domain of the defined construct are linked with each other. An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80). An envisaged peptide linker with less than 5 amino acids can comprise 4, 3, 2 or one amino acids. A particularly preferred “single” amino acid in context of the “peptide linker” is Gly. Accordingly, the peptide linker may consist of one or more Gly residues. Furthermore, peptide linkers that also do not promote any secondary structures are preferred. The linkage of the domains to each other can be provided by, e.g., genetic engineering. Methods for preparing fused and operatively linked bispecific single chain constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001).

The bispecific single chain antibody constructs described herein above and below may be humanized or deimmunized antibody constructs. Methods for the humanization and/or deimmunization of (poly)peptides and, in particular, antibody constructs are known to the person skilled in the art.

In one embodiment of the pharmaceutical composition of this disclosure, the V_{H} and V_{L} regions of a human CD3 specific domain are derived from a CD3 specific antibody selected from the group consisting of X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, WT31 and F101.01. These CD3-specific antibodies are well known in the art and, inter alia, described in Tunnacliffe (1989), Int. Immunol. 1, 546-550. In specific embodiments, V_{H} and V_{L} regions are derived from antibodies/antibody derivatives and the like that are capable of specifically recognizing the human CD3-ε chain or human CD3-ζ chain.

VIII. Polynucleotides Encoding Chemotherapy Resistance Moiety and/or Antigen Recognition Moiety

The present disclosure encompasses a composition comprising a nucleic acid sequence encoding a chemotherapy resistance moiety, antigen recognition moiety, or both, as defined herein and cells harboring the nucleic acid sequence. The nucleic acid molecule is a recombinant nucleic acid molecule, in particular aspects and may be synthetic. It may comprise DNA, RNA as well as PNA (peptide nucleic acid) and it may be a hybrid thereof.

It is evident to the person skilled in the art that one or more regulatory sequences may be added to the nucleic acid molecule comprised in the composition of the disclosure. For example, promoters, transcriptional enhancers and/or sequences that allow for induced expression of the polynucleotide of the disclosure may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, it is envisaged for further purposes that nucleic acid molecules may contain, for example, thioester bonds and/or nucleotide analogues. The modifications may be useful for the stabilization of the nucleic acid molecule against endo- and/or exonucleases in the cell. The nucleic acid molecules may be transcribed by an appropriate vector comprising a chimeric gene that allows for the transcription of said nucleic acid molecule in the cell. In this respect, it is also to be understood that such polynucleotides can be used for “gene targeting” or “gene therapeutic” approaches. In another embodiment the nucleic acid molecules are labeled. Methods for the detection of nucleic acids are well known in the art, e.g., Southern and Northern blotting, PCR or primer extension. This embodiment may be useful for screening methods for verifying successful introduction of the nucleic acid molecules described above during gene therapy approaches.

The nucleic acid molecule(s) may be a recombinantly produced chimeric nucleic acid molecule comprising any of the aforementioned nucleic acid molecules either alone or in combination. In specific aspects, the nucleic acid molecule is part of a vector.

The present disclosure therefore also relates to a composition comprising a vector comprising the nucleic acid molecule described in the present disclosure.

Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells. A cloning vector may be used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

In specific embodiments, there is a vector that comprises a nucleic acid sequence that is a regulatory sequence operably linked to the nucleic acid sequence encoding a bispecific single chain antibody constructs defined herein. Such regulatory sequences (control elements) are known to the artisan and may include a promoter, a splice cassette, translation initiation codon, translation and insertion site for introducing an insert into the vector. In specific embodiments, the nucleic acid molecule is operatively linked to said expression control sequences allowing expression in eukaryotic or prokaryotic cells.

It is envisaged that a vector is an expression vector comprising the nucleic acid molecule encoding a chemotherapy resistance moiety and/or antigen recognition moiety as defined herein. In specific aspects, the vector is a viral vector, such as a lentiviral vector. Lentiviral vectors are commercially available, including from Clontech (Mountain View, Calif.) or GeneCopoeia (Rockville, Md.), for example.

The term “regulatory sequence” refers to DNA sequences that are necessary to effect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is an expression vector, in certain embodiments. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the P_{L}, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements that are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al. Cancer Immunol Immunother (2001) 50(3), 141-150) or pSPORT1 (GIBCO BRL).

In some embodiments, the expression control sequences are eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the disclosure may follow.

Additional regulatory elements may include transcriptional as well as translational enhancers. Advantageously, the above-described vectors of the disclosure comprises a selectable and/or scorable marker. Selectable marker genes useful for the selection of transformed cells are well known to those skilled in the art and comprise, for example, antimetabolite resistance as the basis of selection for dhfr, which confers resistance to methotrexate (Reiss, Plant Physiol. (Life-Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus which confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule can be used in a cell, alone, or as part of a vector to express the encoded polypeptide in cells. The nucleic acid molecules or vectors containing the DNA sequence(s) encoding any one of the above described bispecific single chain antibody constructs is introduced into the cells that in turn produce the polypeptide of interest. The recited nucleic acid molecules and vectors may be designed for direct introduction or for introduction via liposomes, or viral vectors (e.g., adenoviral, retroviral) into a cell. In certain embodiments, the cells are T-cells, CAR T-cells, NK cells, NKT-cells, MSCs, neuronal stem cells, or hematopoietic stem cells, for example.

In accordance with the above, the present disclosure relates to methods to derive vectors, particularly plasmids, cosmids, viruses and bacteriophages used conventionally in genetic engineering that comprise a nucleic acid molecule encoding the polypeptide sequence of a bispecific single chain antibody constructs defined herein. Preferably, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the recited polynucleotides or vector into targeted cell populations. Methods which are well known to those skilled in the art can be used to construct recombinant vectors; see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the disclosure can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

A. Vectors

An expression vector comprising coding sequence for a chemotherapy resistance moiety and/or coding sequence for an antigen recognition moiety is encompassed in the disclosure. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

B. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the disclosure, the use of internal ribosome entry sites (IRES) elements are used to create multigene, or polycistronic, messages, and these may be used in the disclosure.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

C. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

D. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present disclosure may be a viral vector that encodes one or more CARs of the disclosure. Non-limiting examples of virus vectors that may be used to deliver a nucleic acid of the present disclosure are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present disclosure as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

E. Other Viral Vectors

Other viral vectors may be employed as vaccine constructs in the present disclosure. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

F. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

G. Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

H. Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present disclosure. In particular aspects, the transplanted cells or tissues may be placed into an organism. In preferred facets, a nucleic acid is expressed in the transplanted cells.

### IX. Combination Therapy

In certain embodiments of the disclosure, the immunotherapy/chemotherapy methods of the present disclosure for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with other therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present disclosure, it is contemplated that the TMZ and cell therapy could be used similarly in conjunction with chemotherapeutic, radiotherapeutic, or immunotherapeutic intervention, and also other pro-apoptotic or cell cycle regulating agents.

Alternatively, the present inventive therapy may precede or follow the other agent treatment by intervals ranging from minutes to weeks. In embodiments where the other agent and present disclosure are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, wherein the present disclosure is “A” and the secondary agent, such as radio- or chemotherapy, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer, or any analog or derivative variant of the foregoing and also combinations thereof.

In specific embodiments, chemotherapy for the individual is employed in conjunction with the disclosure, for example before, during and/or after administration of the disclosure.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a gene therapy in which a therapeutic polynucleotide is administered before, after, or at the same time as the present disclosure clinical embodiments. A variety of expression products are encompassed within the disclosure, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present disclosure to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-lbeta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Combining TMZ with HER2.CAR T Cells Leads to Enhanced Tumor Killing

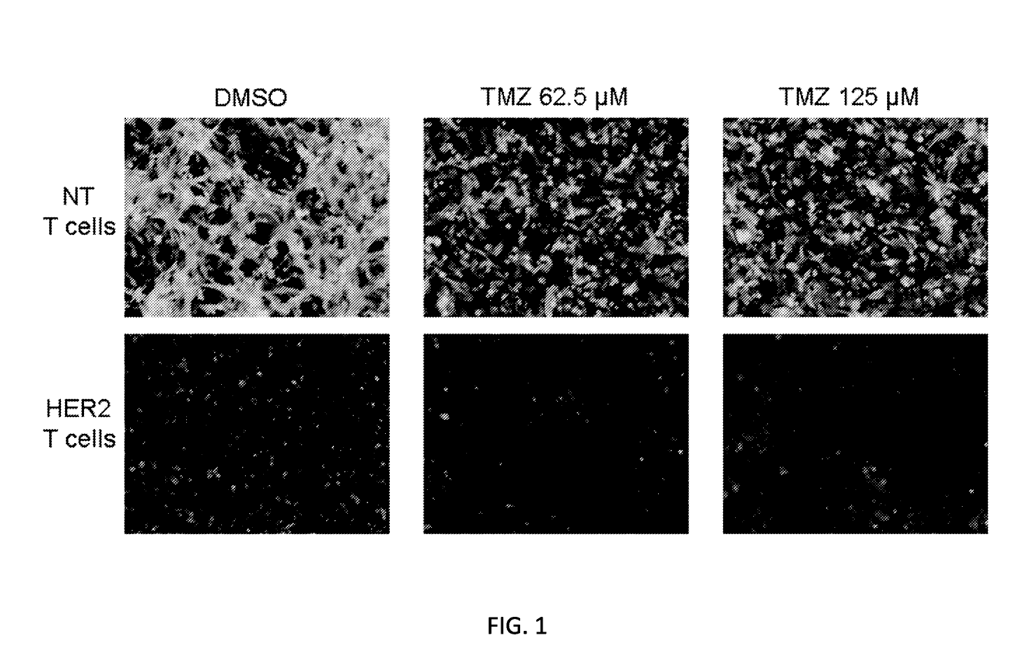

Both TMZ and HER2.CAR T cells have anti-GBM activity on their own; however, it is unknown whether combining the agents provides additional benefit. To test this, U373.eGFP.FFLuc cells were co-cultured with either non-transduced (NT) or HER2.CAR T cells with or without TMZ at an effector-to-target ratio that resulted in incomplete tumor killing by the T cells alone. NT-T cells in the absence of TMZ had no antitumor activity (FIG. 1). HER2.CAR T cells had limited antitumor activity in the absence of TMZ, similar to TMZ with NT-T cells. However, the groups receiving both TMZ and HER2.CAR T cells showed complete tumor killing, indicating that chemoimmunotherapy for GBM is beneficial (FIG. 1).

### Example 2

### Inhibits T-Cell Expansion

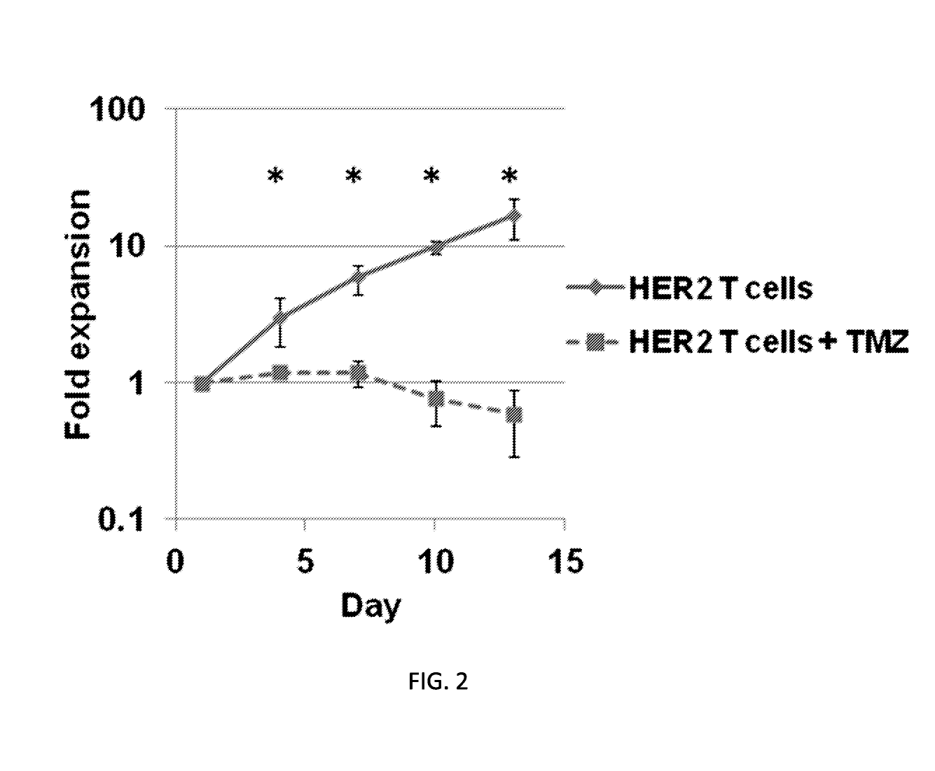

While it was shown that combining TMZ with HER2.CAR T cells can enhance tumor cell killing, it was desired to determine the effect of TMZ on the T cells themselves, specifically the effect of TMZ on T-cell expansion. To do this, 1×10⁶ HER2.CAR T cells were plated with 100 U/ml of IL-2 and treated with 100 μM TMZ (day 1). Every three days, the T cells were counted, given fresh IL-2, and re-treated with TMZ. While HER2.CAR T cells expanded in the absence of TMZ, treatment with TMZ effectively prevented T-cell expansion in vitro (FIG. 2; p<0.05). This effect may limit the efficacy of T cells adoptively transferred during TMZ therapy, thus providing the rationale for generating HER2.CAR T cells that are TMZ-resistant.

### Example 3

### Generating TMZ-Resistant HER2.CAR T Cells

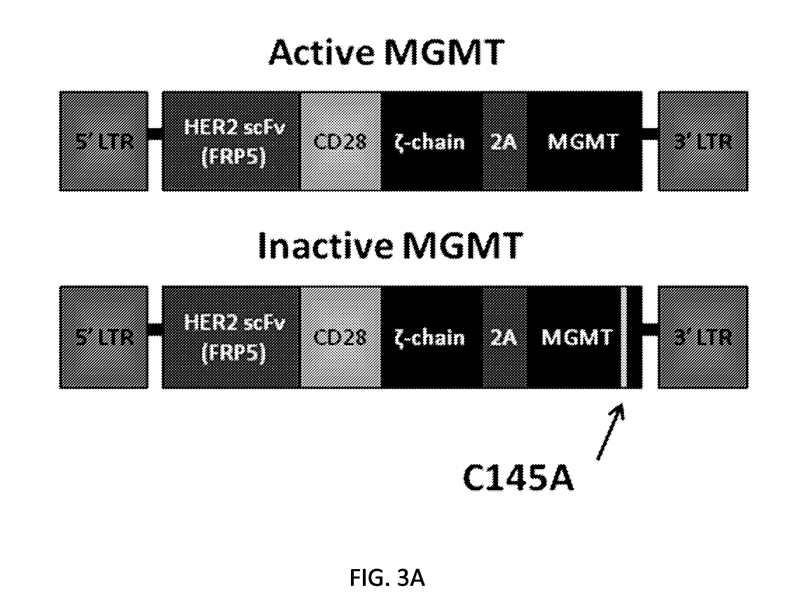

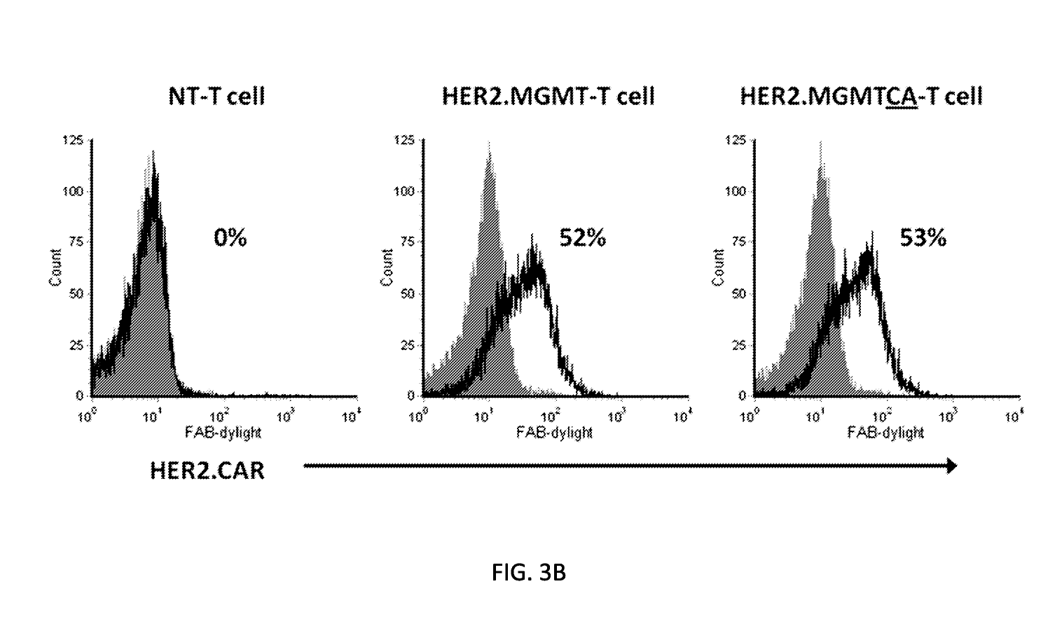

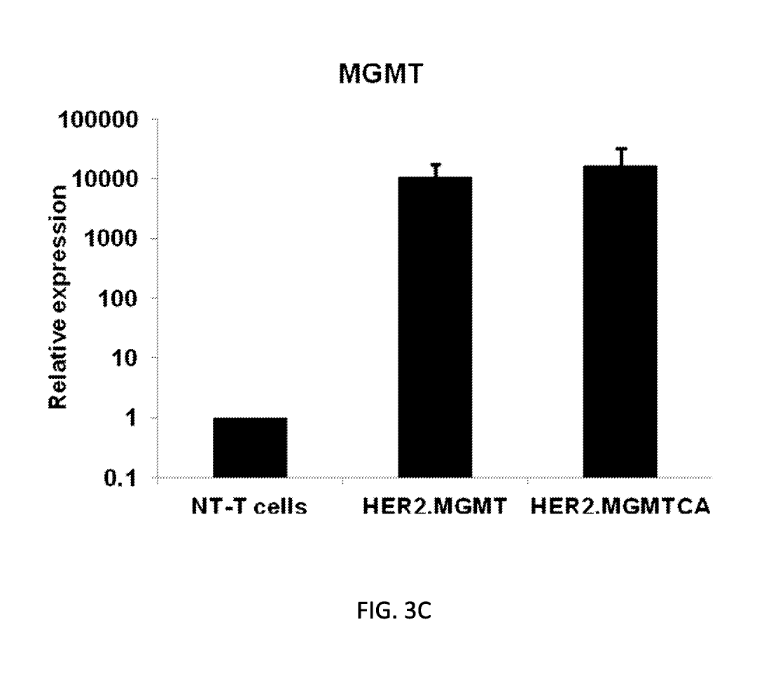

MGMT was cloned into an SFG retroviral vector with a second generation HER2.CAR containing CD28 and CD3-ζ signaling domains (FIG. 3). The two genes were linked by a 2A sequence that allows for the generation of two separate protein products from one vector. This HER2.MGMT construct was used to generate T cells that expressed both the HER2.CAR and functional MGMT protein (HER2.MGMT T cells). A second construct containing a cysteine to alanine amino acid substitution in the MGMT sequence, which has been shown to render the protein inactive, was used to generate control T cells that express the HER2.CAR, and nonfunctional MGMT protein (HER2.MGMTCA T cells; FIG. 3). RD114-pseudotyped retroviral particles encoding these constructs were used to transduce CD3/CD28 activated T cells from normal healthy donors. FACS analysis was used to determine the cell surface expression of the HER2.CAR, while quantitative RT-PCR was used to determine MGMT mRNA expression within the cell. Both HER2.MGMT and HER2.MGMTCA T cells express comparable levels of HER2.CAR and MGMT mRNA (FIG. 3).

### Example 4

### T Cells Transduced with Active MGMT Expand in the Presence of TMZ

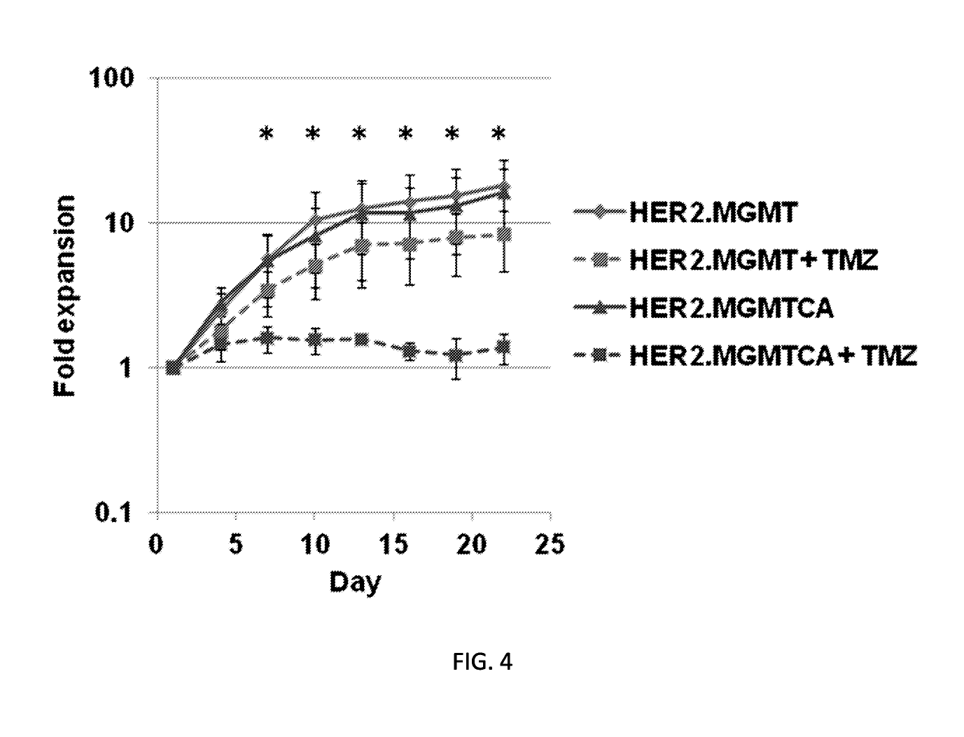

To determine if the expression of active MGMT can rescue T cells from the lymphotoxic effects of TMZ, the T-cell expansion studies were repeated with the HER2.MGMT T cells. 1×10⁶ HER2.MGMT T cells were plated on day 1 with 100 U/ml of IL-2 and treated with 100 μM TMZ. Every three days, the T cells were counted, given fresh IL-2, and re-treated with TMZ. HER2.MGMTCA T cells cultured in parallel served as a control. While both HER2.MGMT and HER2.MGMTCA T cells expanded in the absence of TMZ, only HER2.MGMT T cells expanded in the presence of TMZ (FIG. 4; p<0.05 for HER2.MGMT+TMZ vs. HER2.MGMTCA+TMZ from day 7 to day 22). The control HER2.MGMTCA T cells with the inactive MGMT behaved similarly to the HER2.CAR T cells shown in FIG. 2 that lacked transgenic MGMT.

### Example 5

### HER2.MGMT T Cells Undergo Less Apoptosis in the Presence of TMZ

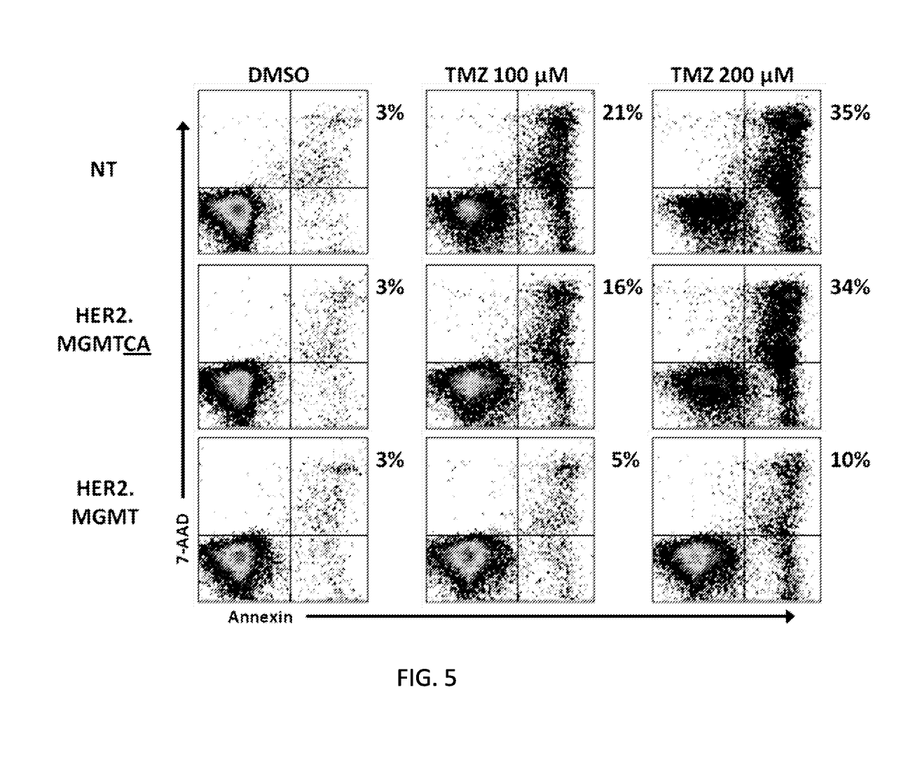

As described above, TMZ works by causing DNA damage that ultimately leads to cell death by the apoptotic pathway. Thus, to further characterize the TMZ-resistance of the genetically modified T cells, they were cultured in the absence or presence of TMZ and stained them for annexin and 7-AAD, markers of apoptotic cell death. As shown in FIG. 5, non-transduced (NT), HER2.MGMTCA, or HER2.MGMT T cells undergo very low and comparable levels of apoptotic cell death in the absence of TMZ. However when treated with increasing amounts of TMZ, HER2.MGMT T cells underwent less apoptosis than NT or HER2.MGMTCA T cells (FIG. 5). Together with the T-cell expansion data, this indicates that the expression of active MGMT effectively confers TMZ-resistance to the T cells.

### Example 6

### Pretreatment of HER2.MGMT T Cells with TMZ does not Impair Tumor Killing

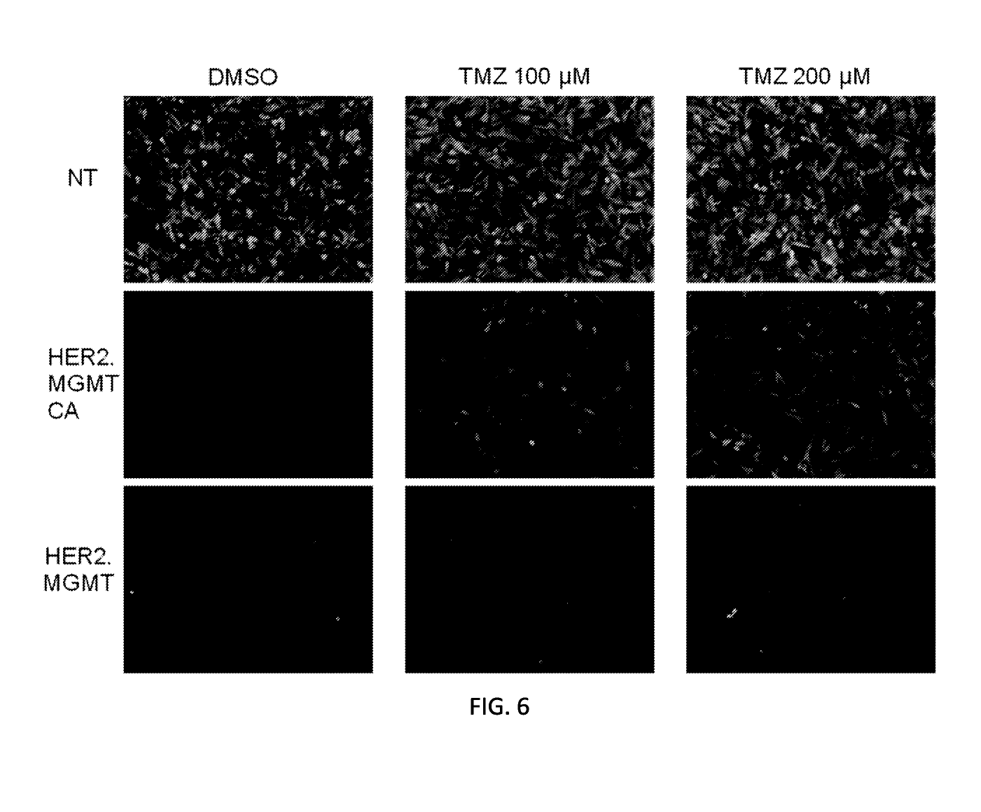

To determine if TMZ-resistance can enhance the ability of T cells to kill tumor cells in the presence of TMZ, 5×10⁵ non-transduced (NT), HER2.MGMTCA, or HER2.MGMT T cells were pretreated with TMZ for 1 week and then challenged with 5×10⁵ U373.eGFP.FFLuc cells. After 48 hours, the T cells were removed and the tumor cells were imaged by fluorescence microscopy. In contrast to HER2.MGMTCA T cells, HER2.MGMT T cells were able to completely kill the tumor cells even after being pretreated with TMZ (FIG. 6). HER2.MGMTCA T cells showed complete tumor killing in the absence of TMZ that was impaired with TMZ pretreatment. NT T cells had no antitumor activity.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A polynucleotide comprising a sequence that encodes O⁶-methylguanine-DNA methyltransferase (MGMT) and a sequence that encodes a chimeric antigen receptor (CAR).

2. The polynucleotide of claim 1, wherein the MGMT and the CAR are expressed as gene products that are separate polypeptides.

3. The polynucleotide of claim 1, wherein the CAR is specific for a tumor antigen.

4. The polynucleotide of claim 1, wherein the CAR is specific for a tumor antigen that is present on a cancer cell, wherein the cancer is treatable by Temozolomide (TMZ).

5. The polynucleotide of claim 3, wherein the tumor antigen is expressed in glioblastoma multiforme (GBM) cells, melanoma cells, lymphoma cells, breast cancer cells, prostate cancer cells, or neuroblastoma cells.

6. The polynucleotide of claim 3, wherein the tumor antigen is any antigen expressed in the tumor and/or associated tumor stroma including HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, viral-associated antigens expressed by the tumor, or other tumor-associated antigens that are identified through genomic analysis and/or differential expression studies of tumors.

7. An expression vector, comprising the polynucleotide of claim 1.

8. The vector of claim 7, wherein the vector is a viral vector selected from the group consisting of a retroviral vector, lentiviral vector, adenoviral vector, and an adeno-associated viral vector.

9. (canceled)

10. A cell comprising the expression vector of claim 7.

11. The cell of claim 10, further defined as an immune system cell selected from the group consisting of a T cell, NK cell, and a NKT cell.

12. (canceled)

13. The cell of claim 10, further defined a T cell.

14. A method of treating cancer that is treatable with TMZ, comprising the step of delivering a therapeutically effective amount of the cells of claim 10 to an individual that is receiving, has received, or will receive TMZ.

15. The method of claim 14, further defined as delivering a therapeutically effective amount of TMZ to the individual.

16. The method of claim 15, wherein the cells and the TMZ are delivered concomitantly.

17. The method of claim 15, wherein the cells and the TMZ are delivered at separate times.

18. (canceled)

19. (canceled)

20. The method of claim 14, wherein the cancer is glioblastoma multiforme, melanoma, lymphoma, breast cancer, prostate cancer, or neuroblastoma.

21. The method of claim 14, wherein the cancer is metastatic cancer.

22. The method of claim 14, wherein the CAR is specific for any antigen expressed in the tumor and/or associated tumor stroma including HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α2, IL-11 receptor R α, MUC1, MUC16, CA9, GD2, GD3, HMW-MAA, CD171, Lewis Y, G250/CALX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, viral-associated antigens expressed by the tumor, or other tumor-associated antigens that are identified through genomic analysis and or differential expression studies of tumors.

23. The method of claim 14, further comprising the step of delivering an additional cancer treatment to the individual, wherein the additional cancer treatment comprises surgery, chemotherapy, immunotherapy, radiation, hormone therapy, or a combination thereof.

24. (canceled)

25. The method of claim 14, wherein when the individual has unmethylated MGMT promoters, the individual is provided with an effective amount of O⁶-benzylguanine (O⁶-BG).

26. The method of claim 14, further comprising the step of diagnosing the cancer of the individual.

27. (canceled)
